(19) United States
(12) Patent Application Publication
(10) Pub. No.: US 2008/0181882 A1
(43) Pub. Date: Jul. 31, 2008

(54) Neurgulin 1 (NRG1) - ErbB4 signaling as a target for the treatment of schizophrenia

(75) Inventor: Chang-Gyu Hahn, Bryn Mawr, PA (US)

Correspondence Address: Pearl Cohen Zedek Latzer, LLP, 1500 Broadway, 12th Floor, New York, NY, 10036, US

(73) Assignee: The University of Pennsylvania

(21) Appl. No.: 11/984,285

(22) Filed: Nov. 15, 2007

Related U.S. Application Data
(60) Provisional application No. 60/858,934, filed on Nov. 15, 2006.

(51) Int. Cl. A61K 39/395; A61K 31/7105; G01N 33/567; A61P 25/18

(52) U.S. Cl. 424/130.1; 514/44; 435/7.21

(57) ABSTRACT

This invention relates to methods and compositions for the treatment of schizophrenia. Specifically, provided herein are methods and compositions for the treatment of schizophrenia by modulating the effect of Neuregulin-1 on the stimulation of erbB and its subsequent effect on schizophrenic prefrontal cortex.

### CROSS REFERENCE TO RELATED APPLICATIONS

This application claims priority from U.S. Provisional Application Ser. No. 60/858,934, filed Nov. 15, 1006, which is incorporated herein by reference in its entirety.

### GOVERNMENT INTEREST

This work was supported in part by US National Institutes of Health Grants No. MH64045 and MH63946. The Government may have certain rights in the invention.

### FIELD OF INVENTION

This invention is directed to methods and compositions for the treatment of schizophrenia. Specifically, provided herein are methods and compositions for the treatment of schizophrenia by modulating the effect of Neuregulin-1 on the stimulation of erbB and its subsequent effect on schizophrenic prefrontal cortex.

### BACKGROUND OF THE INVENTION

Schizophrenia, affects approximately 2 million Americans. At any particular time, about 20% of the hospital beds in the U.S. are occupied by schizophrenic patients. The illness usually develops between adolescence and age 30 and is characterized by positive symptoms (delusions or hallucinations), negative symptoms (blunted emotions and lack of interest) and disorganized symptoms (confused thinking and speech or disorganized behavior and perception). Additionally, cognitive deficits are also frequently observed, particularly in elderly schizophrenia patients (Purohit et al., 1993, Biol. Psychiatry 33(4):255 260). For some patients, the disorder is lifelong, while others may have periodic episodes of psychosis.

Several putative schizophrenia susceptibility genes have been identified. Genomewide linkage studies and metaanalyses of linkage scans have highlighted chromosome 8p as a susceptibility locus. Extensive fine-mapping of the 8p locus, haplotype-association analysis, and linkage disequilibrium (LD) tests subsequently implicated neuregulin 1 (NRG1), a gene with pleotropic roles in neurodevelopment and plasticity.

Recent molecular genetics studies implicate neuregulin 1 (NRG1) and its receptor erbB in the pathophysiology of schizophrenia. Among NRG1 receptors, erbB4 is of particular interest because of its crucial roles in neurodevelopment and in the modulation of N-methyl-Daspartate (NMDA) receptor signaling.

NRG1-mediated erbB signaling has important roles in neural and glial development, as well as in the regulation of neurotransmitter receptors thought to be involved in the pathophysiology of schizophrenia. ErbB4 is of particular interest in relation to the pathophysiology of schizophrenia because erbB4 signaling can modulate neurobiological processes often disturbed in the disorder: neuronal migration, the biology of GABAergic interneurons and NMDA receptor (NMDAR) transmission. Attempts have been made to examin the expression of NRG1 mRNAs in postmortem prefrontal cortex of schizophrenic subjects, with variable results: an overall increase, an increase in type I mRNA or subtle changes in the ratio of type II/type I or type II/type III mRNA (R. Navon et al., Abstr. XIIth World Congr. Psychiatr. Genet. P8.20, 2004; J. Law et al., Soc. Neurosci. Abstr. 109.7, 2004; and ref. 17, respectively). To date, however, no specific role for NRG1 has been established in schizophrenia.

Therefore a need still remains in the art for an effective, and long lasting treatment of the symptoms of schizophrenia, without serious side effects, with future treatment regimes and drug development efforts requiring a more sophisticated approach focused on genetic causes and their modulation.

### SUMMARY OF THE INVENTION

In one embodiment, the invention provides a method of assessing erbB4 signaling in a prefrontal cortex of a schizophrenic subject, comprising the step of stimulating postmortem brain tissues of said subject with an effective amount of Neuregulin-1 (NRG1), thereby enhancing tyrosine phosphorylation of erbB4.

In another embodiment, provided herein is a method of treating schizophrenia in a subject, comprising administering to said subject an agent capable of inhibiting the function of a Neurgulin-1 gene in said subject, whereby said Neurgulin-1 gene stimulates erbB4 signaling.

In one embodiment, provided herein is a composition for the treatment of schizophrenia in a subject, comprising an agent capable of inhibiting the function of a Neurgulin-1 gene in said subject, whereby said Neurgulin-1 gene stimulates ErbB4 signaling which further attenuates NMDAR hypofunction.

In another embodiment, provided herein is a method of screening for an agent capable of postmortem stimulation of a brain tissue, comprising the steps of: slicing frozen brain tissue; gradually thawing the frozen tissue; preparing a cell extract of the sliced tissue; contacting the cell extract with the agent; and immnuopercipitating the agent, wherein immunoblotting will indicate the ability of the agent to stimulate the tissue.

### BRIEF DESCRIPTION OF THE DRAWINGS

FIG. 1 shows that expression of NRG1 or erbB4 proteins is not altered in the PFC of subjects with schizophrenia (SCZ). PFC tissues of control (CTRL) and SCZ subjects were separated for cytosolic and membranous fractions, to assess the expression of NRG1 and erbB4 proteins. (a) NRG1 immunoblotting of slices from control and SCZ subjects. NRG1-specific antibody identified four main bands (at 140 kDa, 110 kDa, 95 kDa and 65 kDa). (b) The main bands were quantified individually and normalized with respect to the signals for b-actin. Between-group differences were not statistically significant. (c) Immunoblotting with erbB4 in slices from control and SCZ subjects. erbB4-specific antibody identified two main bands (at 180 kDa and 85 kDa) that were blocked by the blocking peptide. (d) Quantification of erbB4 proteins in control and SCZ groups showed no significant differences for either the 185-kDa or the 80-kDa bands. Data are presented as mean ±s.e.m. n ¼ 14 for each group;

FIG. 2 shows NRG1-induced erbB4 activation is increased in the PFC of SCZ subjects. (a) PFC slices from control (CTRL) and schizophrenic (SCZ) subjects were stimulated with or without NRG1. Tissue lysates were immunoprecipitated for erbB4 and then immunoblotted with an antibody to phosphotyrosine or erbB4. (b) Scatter plot of pY-erbB4/erbB4 ratios. (c) Immunoblot showing enhanced ERK-2 and AKT activation in PFC of SCZ group after NRG1 stimulation. Tissue lysates were immunoprecipitated for ERK-2 or AKT and then immunoblotted with antibodies to the indicated molecules. (d) Scatterplot showing percent increase in ERK2 and AKT activation in SCZ group with respect to matched controls. pY-ErbB4, phosphotyrosine erbB4; pY/pT-ERK2, phosphotyrosine/threonine ERK2; pS-AKT, phosphoserine AKT;

FIG. 3 shows association of erbB4 with PSD-95 is enhanced in the PFC of schizophrenic subjects. (a) PFC slices were incubated with or without NRG1 for 30 min. Tissue lysates were immunoprecipitated for erbB4 and then probed with PSD-95-specific antibody or erbB4-specific antibody. (b) The ratios of PSD-95 to erbB4 densitometric signals were plotted for the control and schizophrenic groups. (c) A representative immunoblot analysis of PSD-95 in the PFC gray matter. (d) Densitometric analysis of PSD-95 in the slices from 14 matched pairs of schizophrenic and control subjects. CTRL, control; SCZ, schizophrenic;

FIG. 4 shows NRG1 attenuation of NMDAR activation is greater in the schizophrenic subjects than in controls. (a) NRG1 treatment attenuated the NMDA-induced enhancement of NMDAR2A tyrosine phosphorylation as well as the recruitment of PIPLC-γ1 by NMDAR1. (b) Densitometric quantification showing that NMDAR activation induced by NMDA+glycine was decreased in the schizophrenic group. (c) NRG1 suppressed NMDAR activation in the schizophrenic group more than it did in the controls. The y-axis represents the ratio of NMDAR activation in the presence of NRG1 to that in the absence of NRG1. NR, NMDAR; pY-NR2A, phosphotyrosine NR2A; PLC-γ1, PIPLC-γ1; WB, western blot; IP, immunoprecipitation. CTRL, control; SCZ, schizophrenic. *P<0.05, #P<0.01;

FIG. 5 shows NRG1 or NMDA stimulation activates intracellular signal transduction in postmortem brains. (A-D). Postmortem PFC tissues were stimulated either with 20 ng/ml of NRG1-GST (a combination of α and β EGF domains) or with 20 ng/ml of GST at 37C for 30 min. (E-H). Tissues were incubated with either 10 μM NMDA+1 μM glycine or with vehicle alone at 37C for 30 min. Synaptosomal extracts were immunoprecipitated with antibodies for erbB4 (A,D,H), ERK2 (B), akt (C), NMDAR2A (E) and NMDAR1 (F,G). Then, the immunoprecipitates were probed with antibodies for epitopes for phosphotyrosine or binding partners as indicated in the figure. −: no ligand stimulation +: ligand stimulation. pYErb: phosphotyrosine, pY/pT ERK2; phosphotyrosine/threonine ERK2, pSakt; phosphoserine akt, pY-NR2AR; phosphotyrosine, nNOS: PLC-γ1; PIPLC-γ1. NR: NMDAR IP: immunoprecipitation EB: immunoblotting;

FIG. 6 shows NRG1 stimulation induces erbB4 activation in mouse brains at varied postmortem intervals. (A-B) Mice were sacrificed and brains were processed immediately (fresh 0 hour), frozen immediately (frozen 0 hour), or were kept in their bodies in a cold room for 5, 10 or 15 hours. Slices of mouse frontal lobes were stimulated with NRG1 as above and synaptosomal extracts were immunoprecipitated for erbB4. Immunoprecipitates were probed for either pY-erbB4 or PSD-95. (A) Immunoblot demonstrating that NRG1 stimulation induces tyrosine phosphorylation as well as PSD-95 coupling of erbB4 in the brains samples of the PMIs of 0, 5, 10 and 15 hours. (B) Densitometric analysis. The ratios of intensities of pY-erbB4 or PSD-95 over erbB4 are plotted at each time point;

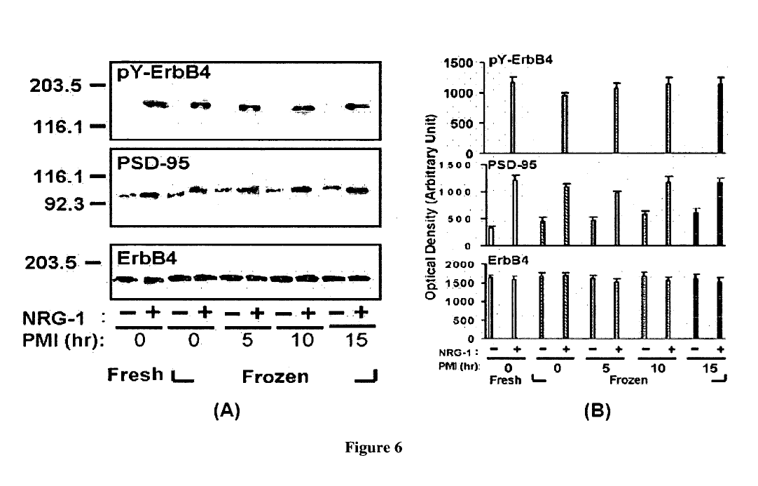

FIG. 7 shows association of erbB4 with NMDAR is enhanced in PFCs of SCZ subjects. (A) Immunoblot demonstrating an increased association of erbB4 with NMDAR1. 50 mm slices of the PFC were incubated with or without NRG 1 for 30 min. Synaptosomal extracts were immunoprecipitated for erbB4 and were probed with anti-erbB4 or anti-NMDAR1. (B) Densitometric analysis of PSD-95 coupling of erbB4. The ratios of intensities of PSD-95 signals to erbB4 were plotted for control (CTRL) and SCZ subjects. Significant differences were noted (t=10.1886, df=13, p<0.001);

FIG. 8 shows association of PSD-95 with NMDAR is enhanced in the PFC of SCZ subjects. PFC slices of 10 matched pairs were immunoprecipitated with antibodies for PSD-95 and probed for erbB4, NMDAR2A or NMDAR1. (A) Immunoblot demonstrating an increased association of PSD-95 with erbB4, NMDAR 1 or NMDAR 2A. Synaptosomal extracts were immunoprecipitated for PSD-95 and were probed with anti-erbB4, anti-NMDAR1, or anti-NMDAR2A. (B) Densitometric analysis of erbB4, NMDAR1, or NMAR2A coupling of PSD-95. The ratios of intensities of the signals over PSD-95 were plotted for control (CTRL) and schizophrenia (SCZ) subjects. (*p<0.001);

FIG. 9 shows haloperidol treatment does not enhance NRG1 induced erbB4 activation. (A) 50 mm slices of PFCs of control and haloperidol treated mice were incubated with or without NRG 1 for 30 min. Tissue lysates were immunoprecipitated for erbB4 and were probed with antiphosphotyrosine or anti-erbB4. (B) Densitometric analysis of erbB4 activation in (A). Graph constructed for the ratios of the intensities for pY-erbB4 over erbB4 for control and haloperidol treated mice. NRG1 induced tyrosine phosphorylation was attenuated in haloperidol treated mice (t(6)=2.49, p=0.46);

FIG. 10 shows ErbB4 expression in human postmortem brains. (A, C) ErbB4 immunoreactivity in the layer III of postmortem PFC (A) and CA1 region of hippocampal formation (C) of a healthy control subject. (C and D) Antibody specificity test showing that erbB4 immunoreactivity can be eliminated by preadsorption of the antibodies (SC-283) with blocking peptides (SC-283-p). Intense erbB4 immunoreactivity was found in both small interneurons and large projection neurons in the prefrontal cortex and the hippocampal formation. This suggests that erbB4 in humans plays a more global role in regulating neuronal activity than it does in rodents. Scale bars: 50 mm;

FIG. 11 shows (I) Various fractions from subcellular fractionation and the PSD fraction were analyzed by immunoblotting for proteins highly enriched in either the PSD or presynaptic vesicles. (II) Electron micrographs of osmicated insoluble pellets obtained by immunopercipitation and Western blotting. A, B: Synaptosomal extracts;

FIG. 12 shows that PSD proteins in synaptosomal or PSD fractions were not dysregulated in the PFC of SCZ subjects compared with matched controls. (A, B) Postmortem PFC tissues from control and SCZ subjects were fractionated and analyzed for proteins by immunoblotting;

FIG. 13 shows that Protein-protein associations were significantly altered in the PSD fractions derived from the PFC of SCZ subjects. (A) A representative immunoblot. (B) Quantification of IP results. (C, D) Synaptosomal fractions of 12 pairs of control and SCZ subjects were immunoprecipitated for PSD-95. (C) A representative immunoblot. (D) Quantification of IP results;

FIG. 14 shows Protein-protein associations indicating trends for dysregulation in PSD fractions of the PFC of SCZ subjects. (A) A representative immunoblot. (B) Quantification of IP results; and

FIG. 15 is a schematic showing (Top) Ligand induced erbB4 signaling is significantly enhanced in the PFC of SCZ subjects, resulting in increased erbB4 activity and decrease NMDAR activity (Bottom) PSD protein-protein interactions are enhanced in SCZ subjects, resulting in erbB4 to PSD-95 and likewise erbB4 to NMDAR-NR1

### DETAILED DESCRIPTION OF THE INVENTION

This invention relates in one embodiment to methods and compositions for the treatment of neuropsychiatric disorders. Specifically, provided herein are methods and compositions for the treatment of neuropsychiatric disorders by modulating the effect of Neuregulin-1 on the stimulation of erbB and its subsequent effect on schizophrenic prefrontal cortex.

In one embodiment, Neuregulin-1 (NRG1) family consists of structurally related proteins containing an epidermal growth factor (EGF)-like domain that specifically activate receptor tyrosine kinases of the erbB family: erbB2, erbB3 and erbB4. These isoforms are divided into three classic groups: type I (previously known as acetylcholine receptor inducing activity, heregulin, or neu differentiation factor), type II (glia growth factor) and type III (cysteine-rich domain containing), which are based on distinct amino termini. Additional NRG1 5′ exons have recently been identified, giving rise putatively to novel NRG1 types IV-VI in the human brain.

In one embodiment, provided herein is a method of assessing erbB4 signaling in a prefrontal cortex of a subject having neuropsychiatric disorders, comprising the step of stimulating postmortem brain tissues of said subject with an effective amount of Neuregulin-1 (NRG1), thereby enhancing tyrosine phosphorylation of erbB4.

In another embodiment, ligand-dependent activation of ErbB receptors results in homo- or heterodimerization, which stimulates receptor trans-phosphorylation on cytoplasmic tyrosine residues, creating binding sites for adaptor or enzymatic proteins. EGF receptor and ErbB4 homodimers are active kinases in the absence of coreceptors and ErbB4 activated in one embodiment, by either homo- or heterodimerization. In one embodiment, ErbB4 expression is necessary to confer on neural progenitor cells the ability to respond to NRG1β1 through migration. In one embodiment, erbB4 receptor contains an extracellular ligand-binding domain of 600-630 amino acids, a single transmembrane α-helix, plus an intracellular domain of ˜600 amino acids that includes the tyrosine kinase and regulatory sequences.

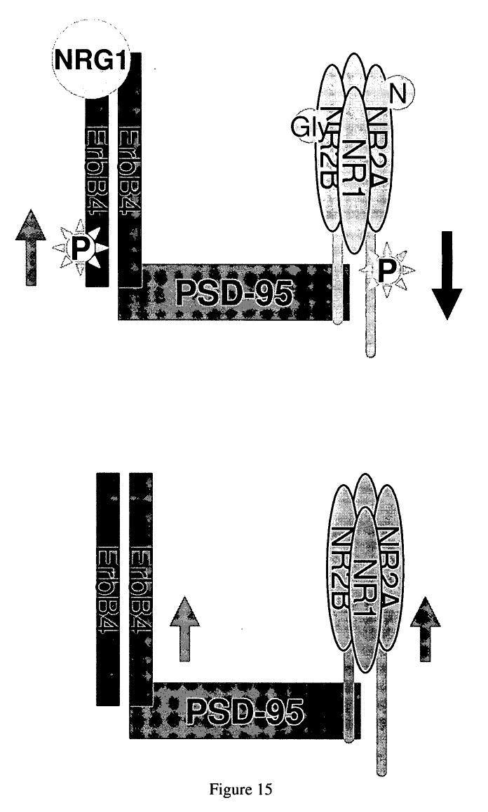

In one embodiment, erbB4 signaling stimulation in the methods and compositions described herein, results in modulation of erbB4-PSD95 binding. In another embodiment, assessing erbB4 signaling in a prefrontal cortex of a schizophrenic subject, comprising the step of stimulating postmortem brain tissues of said subject with an effective amount of Neuregulin-1 (NRG1), thereby enhancing tyrosine phosphorylation of erbB4, further comprises attenuating NMDAR function (See e.g. FIG. 15).

In one embodiment, provided herein is a method of stimulating erbB4 signaling in a prefrontal cortex of a schizophrenic subject, comprising the step of contacting glia, neurons or both in the PFC of the schizophrenia subject with an effective amount of Neuregulin-1 (NRG1), thereby enhancing tyrosine phosphorylation of erbB4. In another embodiment erbB4 signaling stimulation by contact with NRG1 ase described herein, results in modulation of erbB4-PSD95 binding. In another embodiment, stimulating erbB4 signaling in a prefrontal cortex of a schizophrenic subject, comprising the step of contacting glia, neurons or both in the PFC of the schizophrenia subject with an effective amount of Neuregulin-1 (NRG1), thereby enhancing tyrosine phosphorylation of erbB4, further comprises attenuating NMDAR function.

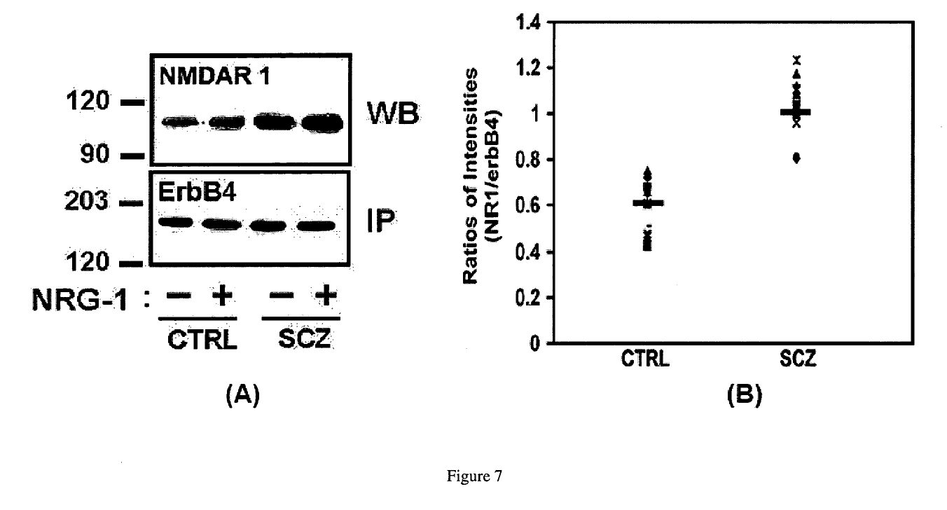

In one embodiment, erbB4-PSD-95 association is distinctly increased in schizophrenia, with PSD-95 protein levels unaltered (FIG. 7), indicating that protein-protein interactions of PSD-95 is important mode of dysregulation in the disease and inhibiting the interaction or binding of erbB4 and PSD95 is effective in controlling the dysregulation of schizophrenia, using the methods described herein.

In one embodiment, schizophrenia is caused by dysregulation of synaptic plasticity in adult subject. ErbB4 receptor is enriched in postsynaptic densities (PSD) and interact with other PSD proteins such PSD-95 (a PDZ domain-containing protein known to aid in receptor scaffolding, interacts primarily with ErbB4 at neuronal synapses where it enhances neuregulin (NRG)-induced kinase activity), NMDA receptor subunit 2C and 2B, Ca2+-activated potassium channels, protein kinase C interacting protein (PICK1) and glutamate (AMPA subtype) receptors.

In another embodiment, NRG1 is implicated in susceptibility to bipolar disorder. In another embodiment, subjects with bipolar disorder who experience predominantly mood-incongruent psychotic features show evidence of an influence of susceptibility from NRG1. In one embodiment, NRG1 is responsible for genome-wide linkage in the 8p12 region (the same chromosome where variation at the neuregulin 1 (NRG1) gene influences susceptibility to schizophrenia), to psychosis in bipolar pedigrees.

In one embodiment, stimulation of erbB4 signaling, caused by contact with NRG1 is pathognomonic of schizophrenia, bipolar disorder or their combination and its attenuation is desirable in the treatment of schizophrenia or bipolar disorder in another embodiment, in subjects exhibiting hyperexpression of NRG1, erbB4 or both. According to this aspect of the invention and in one embodiment, provided herein is method of treating neuropsychiatric disorders, such as schizophrenia or bipolar disorder in certain discrete embodiments in a subject, comprising administering to said subject an agent capable of inhibiting the function of a Neurgulin-1 gene or its encoded or regulated proteins in said subject, whereby said Neurgulin-1 gene stimulates erbB4 signaling.

In another embodiment, inhibiting the function of a Neurgulin-1 (NRG1) gene or its encoded proteins, such as the modulating of erbB pathway, using the agents used in the methods and compositions provided herein, comprises lowering the level of a protein or a nucleic acid regulating the function of said Neurgulin-1 (NRG1) gene, or its encoded or regulated proteins.

In one embodiment, the agent used in the methods and compositions provided herein for inhibiting the function of a Neurgulin-1 gene or its encoded or regulated proteins, is a siRNA, polyamides, triple-helix-forming agents, antisense RNA, synthetic peptide nucleic acids (PNAs), agRNA, LNA/DNA copolymers, small molecule chemical compounds, or a combination thereof.

“Treating” or “treatment” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term. Therefore, in one embodiment, the invention provides a method of treating schizophrenia or bipolar disorder in another embodiment, in a subject, comprising administering to said subject an agent capable of inhibiting the function of a Neurgulin-1 gene or its encoded or regulated proteins in said subject, whereby said Neurgulin-1 gene stimulates erbB4 signaling.

In one embodiment, the term “siRNA” refers to RNA interference, which in another embodiment refers to the process of sequence-specific post-transcriptional gene silencing in animals, mediated by short interfering RNAs (siRNAs). In another embodiment, the process of post-transcriptional gene silencing is an evolutionarily-conserved cellular defense mechanism used to prevent the expression of foreign genes. Such protection from foreign gene expression evolved in one embodiment, in response to the production of double-stranded RNAs (dsRNAs) derived from viral infection or in another embodiment, from the random integration of transposon elements into a host genome via a cellular response that specifically destroys homologous single-stranded RNA of viral genomic RNA. In one embodiment, the presence of dsRNA in cells triggers the RNAi response.

In one embodiment, the term “conserved”, refers to amino acid sequences comprising the peptides or nucleotides described herein, which remain in one embodiment, essentially unchanged throughout evolution, and exhibit homology among various species producing the protein.

The presence of long dsRNAs in cells stimulates in another embodiment, the activity of a ribonuclease III enzyme referred to as dicer. Dicer is involved in one embodiment, in the processing of the dsRNA into short pieces of dsRNA known as short interfering RNAs (siRNAs). Short interfering RNAs derived from dicer activity are in another embodiment about 21 to about 23 nucleotides in length and comprise about 19 base pair duplexes. Small RNAs function in one embodiment, by base-pairing to complementary RNA or DNA target sequences. When bound to RNA, small RNAs trigger RNA cleavage in another embodiment, or translational inhibition of the target sequence in another embodiment. When bound to DNA target sequences, small interfering RNAs mediate in one embodiment, DNA methylation of the target sequence. The consequence of these events, in one embodiment, is the inhibition of gene expression, which, in another embodiment is the NRG1 gene encoding the neuregulin-1 protein described herein. In one embodiment, the agent used for reducing the level or function of NRG1 gene or its encoded protein, is a siRNA specific for the nucleic acide encoding NRG1.

In one embodiment, the siRNA of the NRG1 gene encoding the neuregulin-1 protein described herein, exhibit substantial complimentarity to its target sequence. In another embodiment, “complementarity” indicates that the oligonucleotide has a base sequence containing an at least 15 contiguous base region that is at least 70% complementary, or in another embodiment at least 80% complementary, or in another embodiment at least 90% complementary, or in another embodiment 100% complementary to an-at least 15 contiguous base region present of a target gene sequence (excluding RNA and DNA equivalents). (Those skilled in the art will readily appreciate modifications that could be made to the hybridization assay conditions at various percentages of complementarity to permit hybridization of the oligonucleotide to the target sequence while preventing unacceptable levels of non-specific hybridization). The degree of complementarity is determined by comparing the order of nucleobases making up the two sequences and does not take into consideration other structural differences which may exist between the two sequences, provided the structural differences do not prevent hydrogen bonding with complementary bases. The degree of complementarity between two sequences can also be expressed in terms of the number of base mismatches present in each set of at least 15 contiguous bases being compared, which may range from 0-3 base mismatches, so long as their functionality for the purpose used is not compromised.

In one embodiment, the siRNA of the NRG1 gene encoding the neuregulin-1 protein described herein is sufficiently complimentary to its target sequence. “Sufficiently complementary” refers in one embodiment to a contiguous nucleic acid base sequence that is capable of hybridizing to another base sequence by hydrogen bonding between a series of complementary bases. In another embodiment, complementary base sequences may be complementary at each position in the base sequence of an oligonucleotide using standard base pairing (e.g., G:C, A:T or A:U pairing) or may contain one or more residues that are not complementary using standard hydrogen bonding (including abasic “nucleotides”), but in which the entire complementary base sequence is capable of specifically hybridizing with another base sequence under appropriate hybridization conditions. Contiguous bases are at least about 80% in one embodiment, or at least about 90% in another embodiment, or about 100% complementary to a sequence to which an oligonucleotide is intended to specifically hybridize in another embodiment. Appropriate hybridization conditions are well known to those skilled in the art, can be predicted readily based on base sequence composition, or can be determined empirically by using routine testing (e.g., See Sambrook et al., Molecular Cloning. A Laboratory Manual, 2^{nd} ed. (Cold Spring Harbor Laboratory Press, Cold Spring Harbor, N.Y., 1989).

In one embodiment, minor groove-binding N-methylpyrrole (Py) and N-methylimidazole (Im) polyamides (peptides) uniquely recognize each of the four Watson-Crick base pairs. Antiparallel pairing of imidazole with pyrrole (Im/Py) recognizes in open embodiment, a G-C base pair, whereas in another embodiment, a Py/Py pair recognizes either an A-T or T-A base pair. The binding constant and sequence-specificity of the Py-Im hairpin polyamides are similar to that of a transcription factor. Therefore, many genes, are silenced in other embodiments, by competitive binding of Py-Im hairpin polyamides to their regulatory sequences. Gene expression is controlled in one embodiment, by a combination of multiple common transcription factors. In one embodiment, inhibition of gene expression through the binding of Py-Im polyamides to regulatory sequences is unique to a specific gene, and contains part of the recognition sequence of the transcription factor together with the unique flanking sequences. In another embodiment, targeting Py-Im polyamide to the coding region is more straightforward when selecting a unique sequence. In one embodiment, the agent used to silence the NRG1 gene in the methods and compositions described herein, is Py-Im polyamide specific for the coding region of NRG1, or to regulatory sequences is unique to NRG1 in another embodiment. In another embodiment, the agent used to silence the NRG1 gene in the methods and compositions described herein, is a synthetic polyamide nucleic acid (PNA) specific for the coding region of NRG1, or to regulatory sequences is unique to NRG1 in another embodiment.

In one embodiment, the polyamides used in the compositions and methods described herein, which, in another embodiment are referred to as “peptide nucleic acid” (PNA) or “synthetic peptide nucleic acids”, is an alkylating Py-Im polyamides that show sequence-specific DNA alkylation. In another embodiment, alkylation of a template strand in the coding region of NRG1, by Py-Im polyamide-cyclopropylpyrroloindole (CPI) conjugates with a vinyl linker results in the production of truncated mRNA, effectively inhibiting transcription of NRG1 in vitro. In one embodiment, Py-Im tetra-hydro-cyclo-propabenzindolone (CBI) conjugates with indole linkers are the alkylating polyamides used as the agent capable of inhibiting the expression or function of NRG1 gene, because indole-CBI has increased chemical stability under acidic and basic conditions.

In one embodiment, oligodeoxynucleotides inhibit cellular transcription by binding to duplex DNA to form a triple helix. Due to the possibility of long-term inhibition of the gene product, oligodeoxynucleotides that can bind duplex DNA have advantages over those that bind mRNA or proteins. These oligodeoxynucleotides are generally called triplex forming oligonucleotides (TFOs). By using DNA-specific TFOs, the inhibition of expression of several cellular genes has been demonstrated, including the oncogene, c-myc, the human immunodeficiency virus-1, the alpha chain of the interleukin 2 receptor, the epidermal growth factor receptor, the progesterone responsive gene and the mouse insulin receptor. In one embodiment, the oligonucleotides used in the methods and compositions described herein, can bind to duplex DNA and form triple helices in a sequence-specific manner and will silence expression or function of NRG1.

In one embodiment, homopyrimidine DNA strand (triplex forming oligonucleotide, TFO) can bind to a homopurine/homopyrimide DNA duplex in the major groove by forming Hoogsteen base pairs with the homopurine strand. The Hoogsteen base pairing scheme mediates sequence specific recognition of the double stranded DNA by the TFO where in one embodiment, an AT base pair is recognized by a T; and a GC base pair by a C that is protonated at N3⁺. In another embodiment, homopurine strands specifically form a DNA triplex in which the AT base pair is contacted by an A; and the GC base pair by a G. In one embodiment, the agent capable of inhibiting the expression or function of NRG1 gene is a triple-helix-forming agents. In another embodiment, the triple-helix-forming agents are olygonucletides. In one embodiment, oligonucleotide-mediated triplex formation prevent transcription factor binding to promoter sites and block mRNA synthesis in vitro and in vivo.

In another embodiment, DNA intercalating or cross-linking agents are used to prolong oligonucleotide-duplex interactions.

In one embodiment, the term “TFO” or “triplex forming oligonucleotide” refers to the synthetic oligonucleotides of the present invention which are capable of forming a triple helix by binding in the major groove with a duplex DNA structure.

In another embodiment, the term “bases” refers to both the deoxyribonucleic acids and ribonucleic acids. The following abbreviations are used, “A” refers to adenine as well as to its deoxyribose derivative, “T” refers to thymine, “U” refers to uridine, “G” refers to guanine as well as its deoxyribose derivative, “C” refers to cytosine as well as its deoxyribose derivative. A person having ordinary skill in this art would readily recognize that these bases may be modified or derivatized to optimize the methods described herein, without changing the scope of the invention.

The term “nucleic acid” as used in connection with siRNA, refers in one embodiment to a polymer or oligomer composed of nucleotide units (ribonucleotides, deoxyribonucleotides or related structural variants or synthetic analogs thereof) linked via phosphodiester bonds (or related structural variants or synthetic analogs thereof). Thus, the term refers to a nucleotide polymer in which the nucleotides and the linkages between them are naturally occurring (DNA or RNA), as well as various analogs, for example and without limitation, peptide-nucleic acids (PNAs), phosphoramidates, phosphorothioates, methyl phosphonates, 2-O-methyl ribonucleic acids, and the like. In one embodiment, the siRNAs used in the compositions and methods of the invention, are nucleic acid sequences.

In one embodiment oligomeric antisense compounds, particularly oligonucleotides, are used in modulating the function of nucleic acid molecules encoding NRG1, ultimately modulating the amount of neuregulin-1 produced. This is accomplished by providing antisense compounds which specifically hybridize with one or more nucleic acids encoding NRG1. As used herein, the terms “target nucleic acid” and “nucleic acid encoding NRG1” encompass DNA encoding NRG1, RNA (including pre-mRNA and mRNA) transcribed from such DNA, and also cDNA derived from such RNA. The specific hybridization of an oligomeric compound with its target nucleic acid interferes in another embodiment, with the normal function of the nucleic acid. The modulation of function of a target nucleic acid by compounds which specifically hybridize to it, is referred to in one embodiment as “antisense”. In one embodiment, the functions of DNA to be interfered with using the antisense oligonucleotides described herein, which are used in the methods and compositions described herein, include replication and transcription. In another embodiment, functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of NRG1. In one embodiment, inhibition of gene expression is preferred and mRNA is a preferred target. In one embodiment, since many genes (including NRG1) have multiple transcripts, “inhibition” also includes an alteration in the ratio between gene products, such as alteration of mRNA splice products.

In one embodiment, specific nucleic acids are targeted for antisense. “Targeting” an antisense compound to a particular nucleic acid, in one embodiment, is a multistep process. The process usually begins with the identification of a nucleic acid sequence whose function is to be inhibited. This may be, for example, a cellular gene (or mRNA transcribed from the gene) whose expression is associated with a particular disorder or disease state, or a nucleic acid molecule from an infectious agent. In one embodiment, the target is a nucleic acid molecule encoding NRG1. The targeting process also includes in another embodiment, determination of a site or sites within this gene for the antisense interaction to occur such that the desired effect, e.g., inhibition of expression of the protein such as neuregulin-1, will result. In one embodiment, an intragenic site is the region encompassing the translation initiation or termination codon of the open reading frame (ORF) of the gene. Since, the translation initiation codon is in one embodiment 5′-AUG (in transcribed mRNA molecules; 5′-ATG in the corresponding DNA molecule), the translation initiation codon is referred to in one embodiment as the “AUG codon,” the “start codon” or the “AUG start codon”. In another embodiment, a minority of genes have a translation initiation codon having the RNA sequence 5′-GUG, 5′-UUG or 5′-CUG, and 5′-AUA, 5′-ACG and 5′-CUG and have been shown to function in vivo. Thus, the terms “translation initiation codon” and “start codon” encompasses in other embodiments, many codon sequences, even though the initiator amino acid in each instance is typically methionine (in eukaryotes) or formylmethionine (in prokaryotes). In another embodiment, “start codon” and “translation initiation codon” refer to the codon or codons that are used in vivo to initiate translation of an mRNA molecule transcribed from a gene encoding NRG1, regardless of the sequence(s) of such codons.

In certain embodiments, a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer in one embodiment, to a portion of such a mRNA or gene that encompasses from about 25 to about 50 contiguous nucleotides in either direction (i.e., 5′ or 3′) from a translation initiation codon. In another embodiment, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” refers in one embodiment to the region between the translation initiation codon and the translation termination codon, is a region which may be targeted effectively. Other target regions include in other embodiments, the 5′ untranslated region (5′UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises in one embodiment, an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region is a preferred target region in one embodiment.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be target regions in one embodiment, and are particularly useful in situations where aberrant splicing is implicated in disease, or where an overproduction of a particular mRNA splice product is implicated in disease in other embodiment, such as schizophrenia or related symptoms like bipolar disorder in another embodiment. Aberrant fusion junctions due to rearrangements or deletions are also preferred targets. In one embodiment, introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect. In one embodiment, the term “hybridization” refers to hydrogen bonding, which may be Watson-Crick, Hoogsteen or reversed Hoogsteen hydrogen bonding, between complementary nucleoside or nucleotide bases. In one embodiment, adenine and thymine are complementary nucleotide bases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, or in the case of in vitro assays, under conditions in which the assays are performed.

Antisense compounds are used in one embodiment, as research reagents and diagnostics. In another embodiment, antisense oligonucleotides, which are able to inhibit gene expression, such as the NRG1 gene, with extreme specificity, are used by those of ordinary skill to elucidate the function of particular genes. Antisense compounds are used in another embodiment, to distinguish between functions of various members of a biological pathway. Antisense modulation has, in one embodiment of the agents described in the methods and compositions described herein, been harnessed for research use.

In another embodiment, the antisense used in the methods and compositions described herein, is a DNA peptide nucleic acid (PNA), phosphorothioate DNA, phosphorodithioate DNA, phosphoramidate DNA, amide-linked DNA, MMI-linked DNA, 2′-O-methyl RNA, alpha-DNA, methylphosphonate DNA, 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, anhydrohexitol DNA, C-5 substituted pyrimidine nucleoside, C-7 substituted 7-deazapurine nucleoside, inosine nucleoside phosphorodiamidate morpholino oligonucleotide (PMO), a locked nucleic acid (LNA) or diaminopurine nucleoside

In one embodiment, the specificity and sensitivity of antisense agents described herein, is also harnessed for therapeutic uses. Antisense oligonucleotides are employed in one embodiment, as therapeutic moieties in the treatment of disease states in animals and man. In one embodiment, antisense oligonucleotides are safely and effectively administered to humans. In one embodiment oligonucleotides are useful therapeutic modalities that can be configured to be useful in treatment regimes of cells, tissues and animals, especially humans. In one embodiment, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

In one embodiment, the oligonucleotides used in the methods and compositions described herein, are synthetic peptide nucleic acids (PNAs) which interact with the nucleotide sequence encoding NRG1 in a sequence-specific manner and silence expression or function of NRG1. In another embodiment, the oligonucleotides used in the methods and compositions described herein, are locked nucleic acid (LNA), which interact with the nucleotide sequence encoding NRG1 forming a LNA/DNA co-polymer, in a sequence-specific manner and substantially silence expression or function of NRG1.

In one embodiment, the term “locked nucleic acid” (LNA) refers to a synthetic nucleic acid analogue, incorporating “internally bridged” nucleoside analogues. Synthesis of LNA, and properties thereof, have been described by a number of authors: Nielsen et al, (1997 J. Chem. Soc. Perkin Trans. 1, 3423); Koshkin et al, (1998 Tetrahedron Letters 39, 4381); Singh & Wengel (1998 Chem. Commun. 1247); and Singh et al, (1998 Chem. Commun. 455). As with PNA, LNA exhibits greater thermal stability when paired with DNA, than do conventional DNA/DNA heteroduplexes. In one embodiment, LNA can be joined to DNA molecules by conventional techniques. Therefore, in one embodiment, LNA is to be preferred over PNA, for use in the agents of the methods and compositions described herein.

In one embodiment, the target specific regions of the agent that is able to inhibit gene expression, such as the NRG1 gene, may comprise LNA and/or PNA and the arm region comprise DNA, with the agent further comprising a destabilizing moiety.

In another embodiment, the agent capable of inhibiting expression or function of NRG1 gene, or its encoded protein is an agPNA. In another embodiment, this antibody is referred to as antigenic PNA.

In one embodiment, the term phosphorodiamidate morpholino oligonucleotide (PMO) refers to a ssDNA analog with a synthetic polymorpholino backbone, to which nucleotide bases are linked through phosphorodiamidate groups. Like PNAs, PMOs do not have any charged phosphate groups, making the binding between as PMO/DNA strands of NRG1 gene used in the methods and compositions described herein, stronger than between DNA/DNA strands due to the lack of electrostatic repulsion at physiological pH.

In one embodiment, the agent used in the methods and compositions provided herein for inhibiting the function of a Neurgulin-1 gene or its encoded or regulated proteins, is an antibody or fragment thereof, specific against the protein encoded by the Neuregulin-1 (NRG1) gene or a protein regulated by the expression of NRG1.

Protein and/or peptide homology for any peptide sequence listed herein may be determined by immunoblot analysis, or via computer algorithm analysis of amino acid sequences, utilizing any of a number of software packages available, via methods well known to one skilled in the art. Some of these packages may include the FASTA, BLAST, MPsrch or Scanps packages, and may employ the use of the Smith and Waterman algorithms, and/or global/local or BLOCKS alignments for analysis, for example.

In one embodiment, the term “antibody” include complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include antibody constant domains. In another embodiment, F(ab)'s lack constant domains which are required for complement fixation. scFvs are composed of an antibody variable light chain (V_{L}) linked to a variable heavy chain (V_{H}) by a flexible linker. scFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies described herein can be monoclonal antibodies (Mab) in one embodiment, or polyclonal antibodies in another embodiment. Antibodies of the invention which are useful for the compositions, methods and contraceptives described herein can be from any source, and in addition may be chimeric. In one embodiment, sources of antibodies can be from a mouse, or a rat, or a human in other embodiments. Antibodies of the invention which are useful for the compositions, methods and contraceptives of the invention have reduced antigenicity in humans, and in another embodiment, are not antigenic in humans. Chimeric antibodies as described herein contain in one embodiment, human amino acid sequences and include humanized antibodies which are non-human antibodies substituted with sequences of human origin to reduce or eliminate immunogenicity, but which retain the binding characteristics of the non-human antibody. In another embodiment, the agent capable of attaching to the proteins expressed by NRG1 or its regulated genes, which is used in the methods and compositions described herein, is the erbB4 antibody SC-348, as well as another polyclonal antibody for erbB4, PSD-95 specific antibody 05494 or their combination. In one embodiment, the agent capable of attaching to the proteins expressed by NRG1 or its regulated genes, which is used in the methods and compositions described herein, is specific for erbB4, PSD95, AKT, ERK2, NMDAR or a combination thereof.

In one embodiment, the agent capable of attaching to the proteins expressed by NRG1 or its regulated genes, which is used in the methods and compositions described herein, is an antibody fragment, which is Fab, Fab′, Fab1, Fab2, Fc or scFv.

In one embodiment, provide herein is a method of treating neuropsychiatric disorders in a subject, comprising administering to said subject an agent capable of inhibiting the function of a Neurgulin-1 gene or its encoded or regulated proteins in said subject, whereby said Neurgulin-1 gene stimulates erbB4 signaling, which further comprises stimulating NMDAR function. In one embodiment, schizophrenia is associated with NMDAR hypofunction. In another embodiment, cross-talk between NRG1-erbB4 signaling and NMDAR function are implicated in schizophrenia. In another embodiment, erbB4 signaling is enhanced in schizophrenia and NRG1 stimulation can further mediate NMDAR hypofunction. In one embodiment, erbB4-mediated suppression of NMDAR signaling is an important mechanism underlying the susceptibility to NMDAR hypofunction in schizophrenia and the methods and compositions described herein may be used in certain embodiments to treat the pathology by extrinsic stimulation of NMDAR.

N-Methyl-D-aspartate receptors (NMDARs) are a subtype of ionotropic glutamate receptors (iGluRs) that serve critical functions in physiological and pathological processes in the nervous system, including neuronal development, plasticity and neurodegeneration (Cull-Candy et al., 2001; Lipton and Rosenberg, 1994). NR1 and NR2A-D subunits co-assemble (Meguro et al., 1992; Monyer et al., 1992) to form conventional NMDARs whose activation requires glycine and glutamate as co-agonists (Kleckner and Dingledine, 1988). In one embodiment, stimulating NMDAR function is done by contacting the NMDAR with Glycine, Glutamate, (2S,2′R,3′R)-2-(2′,3′-dicarboxycyclopropyl)glycine (DCG-IV), (2S,1′S,2′S)-2-(carboxycyclopropyl)-glycine (L-CCG-I), Dopamine D₁ receptor agonist SKF81297 NMDA or their combination.

In one embodiment, provide herein is a method of treating neuropsychiatric disorders, such as scizophrenia or bipolar disorder in certain embodiments in a subject, comprising administering to said subject an agent capable of inhibiting the function of a Neurgulin-1 gene or its encoded or regulated proteins in said subject, whereby said Neurgulin-1 gene stimulates erbB4 signaling, which further comprises inhibiting the binding of erbB4 and PSD95. In one embodiment, erbB4-PSD-95 association is distinctly increased in schizophrenia, with PSD-95 protein levels unaltered (FIG. 7), indicating that protein-protein interactions of PSD-95 is important mode of dysregulation in the disease.

In one embodiment, the term “treatment”, or “treating” refers to any process, action, application, therapy, or the like, wherein a subject, including a human being, is subjected to medical aid with the object of improving the subject's condition, directly or indirectly. The term “treating” refers also to reducing incidence, or alleviating symptoms, eliminating recurrence, preventing recurrence, preventing incidence, improving symptoms, improving prognosis or combination thereof in other embodiments.

In another embodiment, treating comprises reducing incidence, inhibiting or suppressing, whereby inhibiting the expression or function of NRG1 gene or its encoded proteins, by the agents used in the methods and compositions described herein, for the treatment of neuropsychiatric disorders, such as scizophrenia or bipolar disorder in certain embodiments, comprises lowering the level of a protein or nucleic acid regulating the expression or function of said NRG1 gene, or inhibiting function of NRG1 gene's encoded proteins. In one embodiment, the agent used in the compositions and methods described herein, is a siRNA, polyamides, triple-helix-forming agents, antisense RNA, synthetic peptide nucleic acids (PNAs), agRNA, LNA/DNA copolymers, small molecule chemical compounds, an antibody or its fragments or a combination thereof. In other embodiments, additional antipsychotic agents are also administered in the methods and are part of the compositions described herein. These include haloperidol and the like.

Recently, a new drug has been found to be effective for treating schizophrenia. This drug, clozapine, is referred to as an atypical antipsychotic. The typical antipsychotic drugs bind to the D2 dopamine receptor and give rise to extrapyramidal side effects through interactions in the nigrostriatal pathways. Clozapine, on the other hand, binds to D3 and D4 dopamine neurons and does not exhibit these side effects. Clozapine does cause systemic side effects in some cases and needs to be monitored closely for these effects. In one embodiment, the methods and compositions described herein further comprise clozapine and its administration. The so-called positive symptoms of schizophrenia that are typified by delusions, hallucinations and formal thought disorder, have proven responsive to treatments with antidopaminergic neuroleptic drugs such as, chlorpromazine, thioridazine and others, which are administered in the methods and compositions of the invention in other embodiments

In one embodiment, the agents described hereinabove are used in the compositions described herein. According to this aspect of the invention and in one embodiment, provided herein is a composition for the treatment of neuropsychiatric disorders, such as scizophrenia or bipolar disorder in certain embodiments in a subject, comprising an agent capable of inhibiting the function of a Neurgulin-1 gene in said subject, whereby said Neurgulin-1 gene stimulates ErbB4 signaling, which further attenuates NMDAR hypofunction.

In one embodiment, provided herein is a method of screening for an agent capable of postmortem stimulation of a brain tissue, comprising the steps of: slicing frozen brain tissue; gradually thawing the frozen tissue; preparing a cell extract of the sliced tissue; contacting the cell extract with the agent; and immnuopercipitating the agent, wherein immunoblotting will indicate the ability of the agent to stimulate the tissue.

In one embodiment the degree to which the methods provided herein can capture in vivo function should be assessed for each signaling mechanism. In another embodiment, multiple parameters are incorporated to properly assess a signaling mechanism, such as tyrosine phosphoryaltion of erbB4 in one embodiment, or erbB4-erbB2 association and activation of ERK or AKT or their combination in other embodiments.

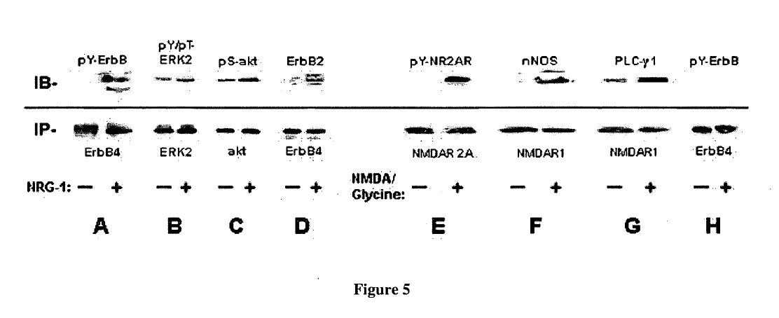

In one embodiment, NRG1 stimulation enhances tyrosine phosphorylation of erbB4 in brains from an almost undetectable basal level. These enhancements are accompanied in another embodiment, by parallel increases in the activation of ERK and AKT, downstream signaling molecules, as well as in the formation of erbB4 and erbB2 heterodimers (FIG. 5) in other embodiments. The results on NRG1 induced erbB4 signaling in postmortem tissues, are predictive in one embodiment of fresh tissues, such as biopsied human glia, adult rodent brain, and cultured cerebellar granule neurons, with respect to tyrosine phosphorylation of erbB4 and other parameters for stimulation induced ErbB4 activation.

In another embodiment, stimulation-induced erbB4 phosphorylation and association of erbB4 with PSD-95 are stable measures in relation to the PMI, while the basal level of phosphorylation appears to be subject to immediate phosphatase activities.

In one embodiment PSD fractions can be isolated from postmortem human brain tissues with the PMI of up to 15 hours. In another embodiment, the expression levels of NR1, NR2A, PSD-95, shank and homer are not dysregulated in synaptosomal extracts as well as in PSD fractions of SCZ subjects. In one embodiment, the association of PSD-95 with erbB4, NR1 and NR2A is enhanced in the PSD fractions of SCZ subjects compared with healthy subjects. In another embodiment, association of NR1 with PSD-95 and NR2A trends toward dysregulations in the PSD fractions of SCZ subjects compared with healthy subjects. In one embodiment, dysregulated protein protein association, is not a direct result of altered partitioning of PSD proteins into the PSD. In another embodiment the levels of expression of PSD proteins in the PSD between SCZ subjects and healthy subjects, is comparable.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

### Postmortem Brain Tissues

All brain tissues were drawn from a prospective clinicopathological studies program at the University of Pennsylvania. The subjects were recruited and longitudinally followed with annual clinical assessments conducted by the Schizophrenia Research Center (see Table 1 for demographic information). The matched controls in this study had no clinical evidence of cognitive or functional decline suggestive of dementia. While none had been prospectively evaluated, detailed histories were used to ascertain their “no cognitive, psychiatric or neurological impairment” status according to standard criteria in use at our Alzheimer's Disease Core Center. Routine diagnostic neuropathological examinations included semiquantitative ratings of vascular lesions, PHF τ-immunoreactive neurofibrillary pathology, β-amyloid plaque deposits, α-synuclein Lewy bodies and dystrophic neurites, gliosis and neuron loss in multiple cortical and subcortical brain regions. Cases exhibiting any abnormal age-related expression of these lesions were excluded from this study as described previously.

TABLE 1
Demographic Information on the Subjects of Postmortem Brains.

| | | | | | Br | | | | Age of | | |
|---|---|---|---|---|---|---|---|---|---|---|---|
| Case | pH | Sex | Race | Age | Wt.^{b} | PMI^{c} | Dementia | MMSE | Onset^{g} | CPZ^{d} Equivalent | |
| 1-CTRL | 6.16 | F | W | 67 | 1100 | 5.5 | no | | | | |
| 1-SCZ | 6.08 | F | W | 67 | 1400 | 8.5 | no | 28 | 16 | 600 | (thiothixene) |
| 2-CTRL | 6.62 | F | W | 74 | 1000 | 3.5 | no | | | | |
| 2-SCZ | 6.34 | F | W | 75 | 1215 | 9 | no | 20 | 16 | 100 | (haloperidol) |
| 3-CTRL | 6.37 | F | W | 85 | 1080 | 11 | no | | | | |
| 3-SCZ | 6.47 | F | W | 81 | 1325 | 13 | no | 21 | 26 | 300 | (thioridazine) |
| 4-CTRL | 6.3 | F | B | 89 | 1000 | 7 | no | | | | |
| 4-SCZ | 6.19 | F | W | 86 | 1100 | 12 | yes | Uncoop^{e} | 19 | 75 | (haloperidol) |
| 5-CTRL | | M | W | 70 | 1388 | 6 | no | | | | |
| 5-SCZ | 6.24 | M | W | 71 | 1270 | 12 | yes | N.D.^{f} | 21 | 1350 | (haloperidol) |
| 6-CTRL | | M | B | 73 | 1249 | 8 | no | | | | |
| 6-SCZ | 6.26 | M | W | 76 | 1322 | 10 | yes | uncoop | 20 | 600 | (thiothixene) |
| 7-CTRL | 6.45 | M | W | 86 | 1202 | 7 | no | | | | |
| 7-SCZ | 6.6 | M | W | 81 | 1385 | 5 | no | N.D. | 19 | 0 | |
| 8-CTRL | 6.5 | M | W | 88 | 1054 | 10 | no | | | | |
| 8-SCZ | 6.42 | M | W | 89 | 1341 | 15 | yes | 5 | 23 | 0 | |
| 9-CTRL | 6.72 | F | W | 91 | 1140 | 11.5 | no | | | | |
| 9-SCZ | 6.48 | F | W | 88 | 1244 | 16 | yes | uncoop | 32 | 0 | |
| 10-CTRL | 6.5 | F | W | 92 | 1107 | 5 | no | | | | |
| 10-SCZ | 6.58 | F | W | 88 | 1200 | 7.5 | no | uncoop | 30 | 0 | |
| 11-CTRL | 6.62 | M | B | 65 | 1346 | 26 | no | | | | |
| 11-SCZ | 6.26 | M | W | 69 | 1503 | 13.5 | no | 24 | 30 | 0 | |
| 12-CTRL | 6.42 | F | B | 81 | 1025 | 8 | no | | | | |
| 12-SCZ | 6.52 | F | W | 76 | 1110 | 9.5 | no | 28 | 23 | 75 | (quetiapine) |
| 13-CTRL | 6.32 | F | B | 83 | 1102 | 22 | no | | | | |
| 13-SCZ | 6.46 | F | W | 82 | 1060 | 12 | no | 20 | 42 | 250 | (clozapine) |

| | | | | | | | | | | |
|---|---|---|---|---|---|---|---|---|---|---|
| 14-CTRL | 6.36 | M | W | 75 | 1390 | 17 | no | | | |
| 14-SCZ | 6.81 | M | W | 75 | 1311 | 12 | no | 19 | 21 | 0 |

These brain tissues were collected from the
^{a}Dx = Diagnosis; N = Normal control; S = Schizophrenia
^{b}Br Wt. = Brain Weight (grams)
^{c}PMI = Post-Mortem Interval (hours)
^{d}CPZ Equivalent = Chlorpromazine equivalent dose (milligrams per day, one month prior to death)
Uncoop^{e} = uncooperative
N.D.^{f} = not done
Age of Onset^{g} = age of onset for schizophrenia

As an index of acidosis associated with agonal states, brain pH on frozen cerebellum was obtained from the cases, which showed no between group differences (matched pairs t=0.16, p=0.87). Furthermore, no correlation was observed between pH and any of the stimulation data.

### Mice.

Adult CH₃ mice were implanted with a Medisorb bioabsorbable polymer disc (Alkermes Inc.), fabricated with haloperidol (n=7) or polymer vehicle only (n=7). These implants were previously shown to release haloperidol for at least 5 months with a calculated release rate of approximately 2 mg/kg per day. After 12 weeks of treatment, mice were sacrificed and the pooled serum haloperidol concentration was measured. Animal experiments were approved by the Institutional and Animal Care and Use Committee of the University of Pennsylvania.

### Tissue Dissection and Fractionation.

1-g blocks of PFC tissue were dissected from fresh frozen coronal brain sections maintained at −80° C. These blocks were derived from Brodmann areas 9, 10 and/or 46. Cytosolic and membranous fractions were isolated as described previously (Wang, H. Y. & Friedman, E. Enhanced protein kinase C activity and translocation in bipolar affective disorder brains. Biol. Psychiatry 40, 568-575 (1996), incorporated herein by reference in its entirety).

Synaptic membrane fractions were isolated by sucrose density gradient, and the PSD extracted in the presence of 1% Triton-X 100 at pH 7.4 (Siekebitz and Philips). Soluble and insoluble fractions were collected and analyzed by immunoblotting and LC-MS/MS.

### Tissue Extracts.

Tissue slices were homogenized in 10 volumes of ice-cold homogenization buffer (25 mM Tris-HCl (pH 7.5), 200 mM NaCl, 2 mM EDTA, 0.5 mM EGTA, 50 μg/ml leupeptin, 0.2 mM phenylmethylsulfonyl fluoride (PMSF), 25 μg/ml pepstatin A, 0.01 U/ml soybean trypsin inhibitor, 5 mM NaF, 1 mM sodium vanadate, 0.5 mM β-glycerophosphate and 0.1% 2-mercaptoethanol) and centrifuged the homogenates at 800 g for 10 min. We solubilized the supernatant with 0.5% digitonin, 0.2% sodium cholate and 0.5% NP-40 for 60 min. We analyzed cleared extracts for immunoblotting.

Stimulation-Induced erbB4 Signaling in Postmortem Brain Tissues.

Postmortem PFC tissues were gradually thawed and sliced using a McIlwain tissue chopper (200 μm×200 μm×3 mm). Tissue slices (50 μm thick) were suspended in ice-cold Krebs-Ringer solution containing 118 mM NaCl, 4.8 mM KCl, 1.3 mM CaCl₂, 1.2 mM KH₂ PO₄, 1.2 mM MgSO₄, 25 mM NaHCO₃, 10 mM glucose, 100 μM ascorbic acid, 50 mg/ml leupeptin, 0.2 mM PMSF, 25 μg/ml pepstatin A and 0.01 U/ml soybean trypsin inhibitor (pH 7.4). We incubated ˜20 mg of tissue with Krebs-Ringer solution containing either 200 ng/ml GST fusion protein or a mixture of neuregulin-1α-GST and neuregulin-1β-GST (200 ng/ml each), at 37° C. for 30 min. During stimulation, the incubation mixture was aerated with 95% O₂, 5% CO₂ every 10 min for 1 min. Ligand stimulation was terminated by adding of 1 ml ice-cold Ca²⁺-free Krebs-Ringer solution containing 0.5 mM EGTA. Tissue slices were harvested by a brief centrifugation and homogenized in 0.25 ml ice-cold immunoprecipitation buffer (same make-up as homogenization buffer described above). The homogenates was centrifuged at 800 g for 10 min and the supernatant sonicated for 10 s. The proteins were solubilized in 0.5% digitonin, 0.2% sodium cholate and 0.5% NP-40 for 60 min. Lysates were cleared by centrifugation at 50,000 g for 5 min and diluted with 0.75 ml of immunoprecipitation buffer. Protein concentrations were determined using the Bradford method (Bio-Rad).

### Immunoprecipitation and Immunoblotting.

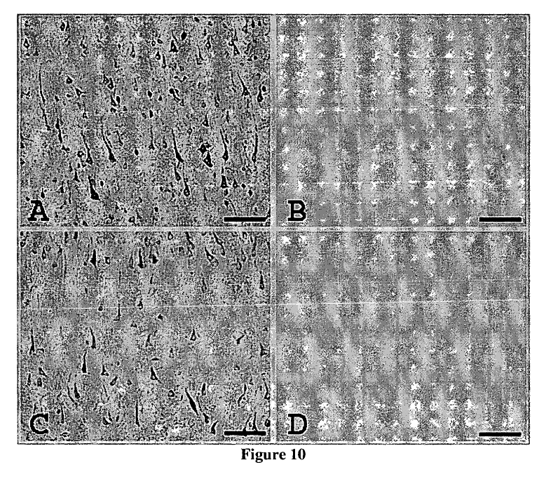

Using 2 μg of anti-erbB4 (SC-283; FIG. 10), 200 μg tissue lysates were immunoprecipitated overnight and then reacted with 50 μl agarose-conjugated protein A or protein G beads (Santa Cruz). erbB4 immunoprecipitates were boiled for 5 min in 100 μl PAGE sample buffer and size-fractionated in 7.5% SDS-PAGE. Immunoblotting was conducted with antibodies for phosphotyrosine (SC-508), erbB2 (SC-7301), NR1 (SC-9058; all from Santa Cruz) and PSD-95 (Upstate, 05494). Blots were stripped and reprobed with erbB4-specific antibody (SC-8050; Santa Cruz). To assess the activation of ERK2 and AKT, brain lysates were immunoprecipitated with antibody to ERK2 (SC-154; Santa Cruz) and antibody to AKT (SC-8312; Santa Cruz), respectively. ERK2 and AKT immunoprecipitates were immunoblotted with antibody to phosophotyrosine ERK (SC-7383; Santa Cruz) and antibody to phosphoserine AKT (SC-7985R; Santa Cruz). The blots were stripped and reprobed with ERK-specific antibody (SC-1647; Santa Cruz) or AKT-specific antibody (Transduction Laboratories, P65920-150), to assess the levels of ERK2 and AKT, respectively.

Subcellular fractions, either synaptosomal or PSD, were immunoprecipitated overnight with anti-NMDAR1 and mixed with of agarose-conjugated protein A/G beads. Solubilized immunoprecipitates were size fractionated, and immuno-blotted with anti-PSD-95, homer, and other binding partners of NMDAR subunits.

Subcellular Fractionation of the PSD from Postmortem Brain Tissues

Synaptic membrane fractions, isolated by sucrose density gradient, were extracted in the presence of 1% Triton-X 100 at pH 6.0 and 7.4 or 8.0 serially (Siekebitz and Philips). Soluble and insoluble fractions were collected and analyzed by either immunoblotting or electron microsocope (EM).

### LC-MS/MS Analysis

Equal amounts of protein from matched pairs were pooled to give 3 SCZ and 3 Control samples. Samples were methanol chloroform extracted, solublized in 2M guanidine, 6M urea and 2M thiourea, reduced, alkylated and trypsin digested. One representative sample was separated by strong cation exchange chromatographydried down, taken up in 30 ul Sol A, and analyzed by LC/LC-MS/MS. Equal amounts of protein from matched pairs were pooled to give 3 SCZ and 3 Control samples. Two peptide ions from selected PSD proteins were monitored in each sample by LC-MS/MS.

### Data Analysis.

Immunoblotting signals were quantified by densitometric scanning and normalized signals for each molecule with respect to β-actin for the analysis of NRG1 or erbB4 expression, or as indicated above. Comparisons between group were conducted for all parameters using linear mixed effects models (NEM) as the general framework to account for the cluster structure due to pair matching and to include the impact of covariates (age, race, sex and PMI). For multiple comparisons, Bonferroni adjustment was implemented (P=0.0019 when adjusted for 26 parameters) and alpha level was set to 0.001. The MEM models were implemented through SAS PROC MIXED. With no covariates, the MEM produces exactly the same results as a paired t-test.

### Example 1

### Stimulation for erbB4 Signaling in Postmortem Brain Tissue

To establish the validity of the stimulation paradigm for erbB4 signaling in postmortem brains, a series of experiments were conducted. First, postmortem tissues were stimulated using NRG1 (α and β EGF domains of NRG1 synthesized as a GST fusion protein, 200 ng/ml, a gift from Dr. Cary Lai) or with GST (200 ng/ml). The tissue extracts were then immunoprecipitated for erbB4 and the erbB4 immune complexes were reacted with anti-phosphotyrosine in an immunoblotting analysis. FIG. 5 A shows that tyrosine phosphorylation was essentially undetectable when tissues were stimulated with GST but was abundant after stimulation with NRG1-GST. Similarly, NRG1 stimulation enhanced ERK2 (FIG. 5 B) and AKT phosphorylation (FIG. 5 C). When activated, erbB4 dimerizes either homodimerizes with another erbB4 or heterodimerizes with erbB2. FIG. 7 shows the result of a co-immunoprecipitation experiment in which association of erbB4 with erbB2 was significantly enhanced following NRG1 stimulation (FIG. 5D). These results on NRG1 induced erbB4 signaling in postmortem tissues, are very much in line with those reported in fresh tissues, such as biopsied human glia, adult rodent brain, and cultured cerebellar granule neurons, with respect to tyrosine phosphorylation of erbB4 and other parameters for stimulation induced ErbB4 activation.

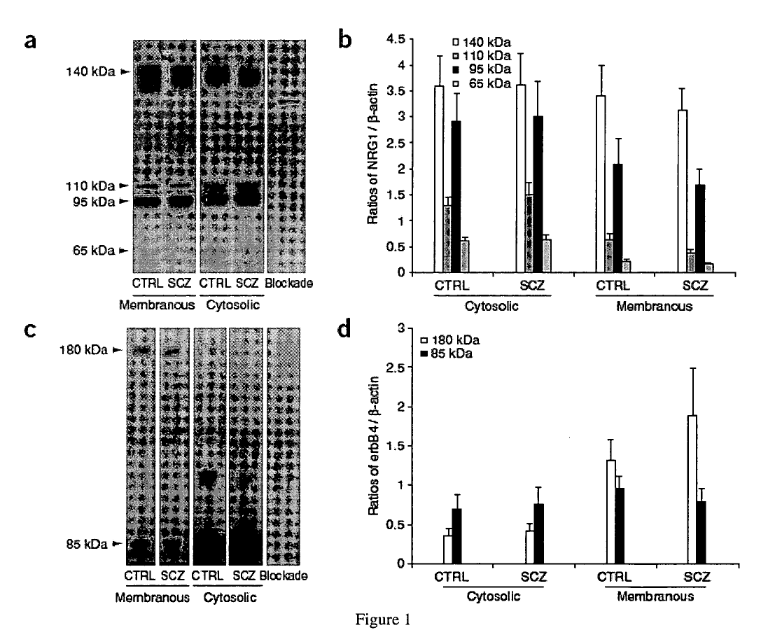

NRG1 and erbB4 proteins were first quantified in postmortem brains using immunoblotting with a polyclonal antiserum (SC-348) that specifically recognizes the ‘a’ type cytoplasmic tail of NRG1 (Frenzel, K. E. & Falls, D. L. Neuregulin-1 proteins in rat brain and transfected cells are localized to lipid rafts. J. Neurochem. 77, 1-12 (2001).). By this method, NRG1 proteins were first quantified in cytosolic and membranous fractions prepared from the dorsolateral prefrontal cortex (PFC). The NRG1-specific antibody identified four major bands, at 65 kDa, 95 kDa, 110 kDa and 140 kDa, in both fractions, all of which were eliminated by preadsorption with antigen peptides (FIG. 1a). Normalized levels of the four bands in the PFC of the schizophrenic subjects were not significantly different from those in the controls (P<0.30; FIG. 1b). Both fractions of PFC were also probed with antibody to erbB4, a polyclonal antiserum specific to the carboxy terminus of human erbB4. Two major bands were identified: a 185-kDa intact molecule and an 80-kDa product of proteolytic cleavage (FIG. 1c). As in the case of NRG1, levels of both erbB4 bands in schizophrenia were not significantly different from control values (P<0.80; FIG. 1d).

### Example 2

### NRG1 Stimulation of erbB4 Signaling Increases Tyrosine Phosphorylation

To establish the validity of the stimulation paradigm for erbB4 signaling in postmortem brains, a series of experiments were conducted. First, postmortem tissues were stimulated using NRG1 (α and β EGF domains of NRG1 synthesized as a GST fusion protein, 200 ng/ml, a gift from Dr. Cary Lai) or with GST (200 ng/ml). The tissue extracts were then immunoprecipitated for erbB4 and the erbB4 immune complexes were reacted with anti-phosphotyrosine in an immunoblotting analysis. FIG. 5 A shows that tyrosine phosphorylation was essentially undetectable when tissues were stimulated with GST but was abundant after stimulation with NRG1-GST. Similarly, NRG1 stimulation enhanced ERK2 (FIG. 5 B) and AKT phosphorylation (FIG. 5 C). When activated, erbB4 dimerizes either homodimerizes with another erbB4 or heterodimerizes with erbB2. FIG. 7 shows the result of a co-immunoprecipitation experiment in which association of erbB4 with erbB2 was significantly enhanced following NRG1 stimulation (FIG. 5D). These results on NRG1 induced erbB4 signaling in postmortem tissues, are very much in line with those reported in fresh tissues, such as biopsied human glia, adult rodent brain, and cultured cerebellar granule neurons, with respect to tyrosine phosphorylation of erbB4 and other parameters for stimulation induced ErbB4 activation.

NRG1 stimulation has enhanced tyrosine phosphorylation of erbB4 in postmortem brains from an almost undetectable basal level. These enhancements were accompanied by parallel increases in the activation of ERK and AKT, downstream signaling molecules, as well as in the formation of erbB4 and erbB2 heterodimers (FIG. 5). In control experiments, the specificity of the NRG1 stimulation protocol was verified for erbB4 signaling by incubating tissues with the control glutathione S-transferase (GST) fusion protein (used for NRG1) alone or with brain-derived neurotrophic factor (BDNF), another trophic factor for a different tyrosine kinase receptor. Neither enhanced the tyrosine phosphorylation of erbB4.

### Example 2

### NRG1-Induced Tyrosine Phosphorylation of erbB4 is Markedly Enhanced in Schizophrenic PFC

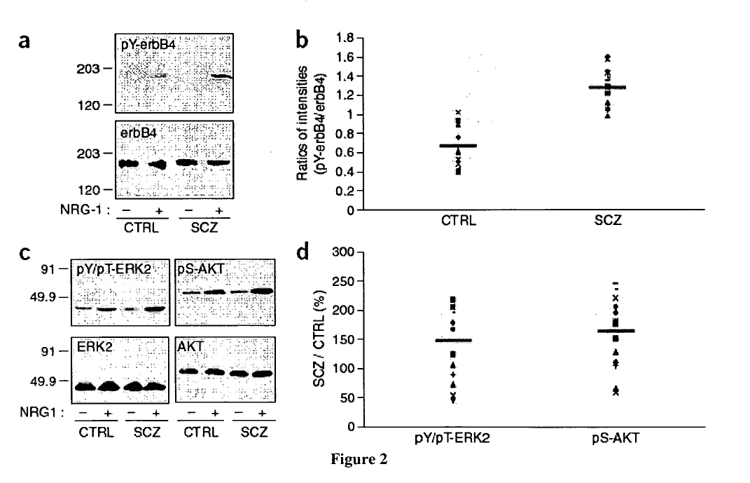

erbB4 signaling was then assessed in the PFC of schizophrenic subjects and matched controls (FIG. 2a). NRG1-induced tyrosine phosphorylation of erbB4 was markedly enhanced in schizophrenic subjects compared to controls (t(13)=8.52, P<0.001; FIG. 2b). Activation of downstream signaling by NRG1 was also enhanced in the schizophrenia group, as indicated by elevated activation of ERK2 (t(13)=6.61, P<0.001) and AKT (t(13)=9.18, P=0.002) in these cases (FIGS. 2c,d). This indicates that enhanced erbB4 signaling in schizophrenia results in downstream cellular effects.

### Example 3

### Demographic and Clinical Variables do not Affect NRG1 Stimulation of erbB4

To assess potential confounding effects of demographic or clinical variables, the levels of NRG1 and erbB4 isoforms were assessed, as well as the activation of erbB4, ERK and AKT, for correlations with sex, age, brain pH, postmortem interval and, for the schizophrenia group, exposure to an antipsychotic drug 1 month before death. No significant correlations were found. To further test whether antipsychotic medication alters erbB4 signaling, the effects of chronic haloperidol treatment were examined in mice at a serum concentration of 3.1 ng/ml for 12 weeks, a level known to induce dopamine D2 receptor upregulation and typical behavioral and physiological changes in mice. NRG1-induced erbB4 activation was significantly reduced in the mice treated with haloperidol compared to those treated with vehicle (t(6)=4.00, P=0.006; FIG. 7).

### Example 4

### Molecular Mechanism

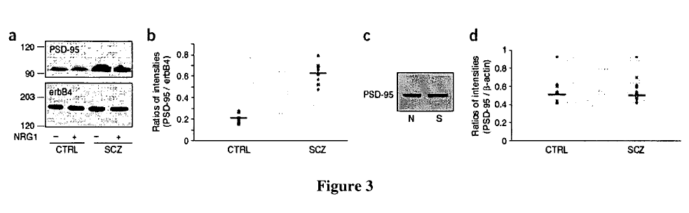

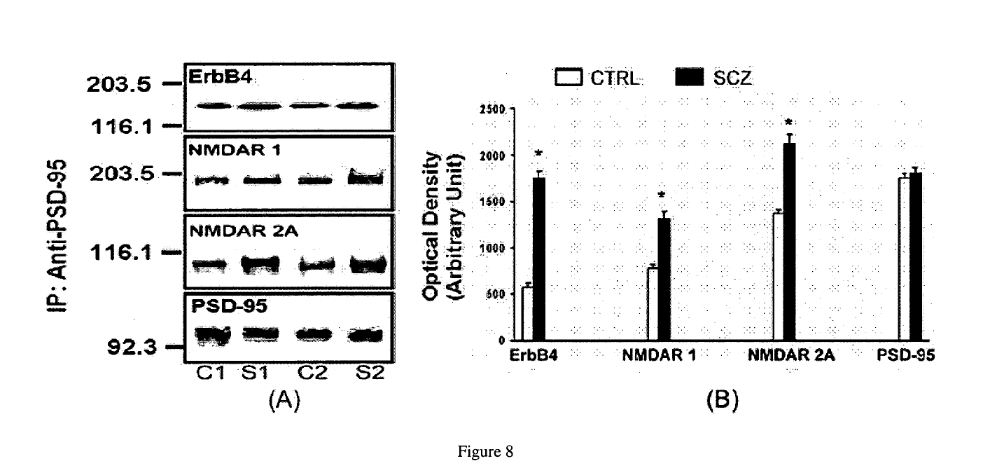

To evaluate the molecular mechanisms of enhanced erbB4 signaling in schizophrenia, we considered erbB4's association with PSD-95, because this protein-protein interaction has a crucial role in the activation of erbB4. PFC tissue lysates were immunoprecipitated for erbB4 and probed with an antibody to PSD-95 (05494). ErbB4 immunoprecipitates contained substantial amounts of PSD-95, indicating a robust association of erbB4 with PSD-95 in postmortem brains (FIG. 3a). Compared to matched controls, the association of erbB4 with PSD-95 was significantly higher in the brains of schizophrenia subjects (t(13)=14.27, P<0.001; FIG. 3b). In addition, the association of erbB4 with NMDAR1 was also increased (FIG. 8). NRG1 stimulation, however, did not increase erbB4-PSD-95 coupling, either in schizophrenia or control subjects, indicating that the enhancement of the erbB4-PSD-95 association in schizophrenia is independent of erbB4 stimulation (FIG. 3a). To determine whether the increased erbB4-PSD-95 association was a result of an increased availability of PSD-95 in schizophrenia, PSD-95 protein expression was measured by immunoblotting. There was no difference between the schizophrenia and control groups (FIGS. 3c, d). Indicating that enhanced erbB4-PSD-95 association is not due to an increased amount of PSD-95, but to an enhanced interaction between the two molecules.

### Example 5

### NRG1 Further Attenuates NMDAR Hypofunction in Schizophrenic PFC

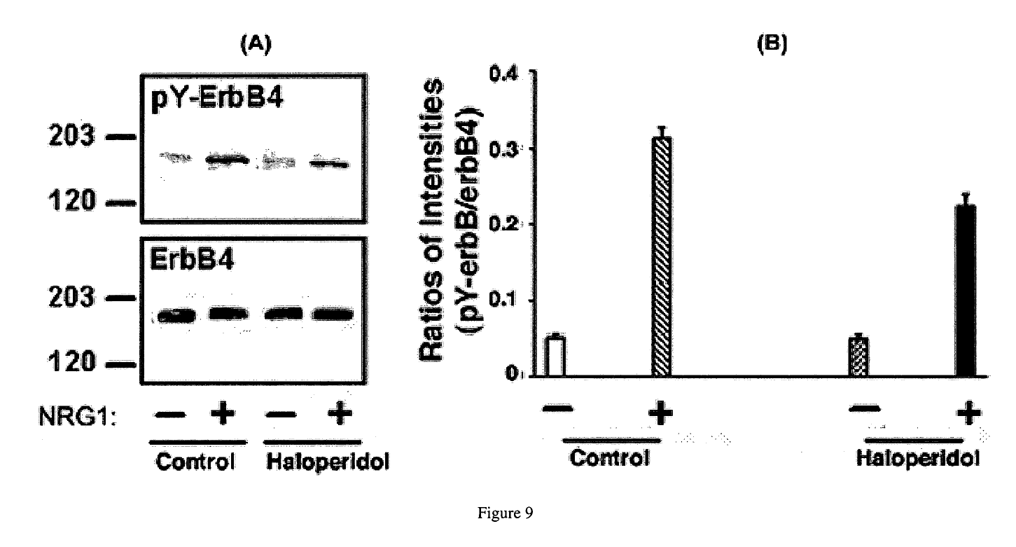

To further validate the experimental paradigm in general and the specificity of stimulation with a given ligand in particular, NMDA stimulation was examined as another receptor system in postmortem tissues. NMDAR hypofunction is a leading hypothesis for explaining the pathophysiology of schizophrenia. Because erbB4 associates with NMDAR via PSD-95, increased coupling of erbB4 with PSD-95 could result in more pronounced effects of NRG1 on NMDAR activation in schizophrenia. To test this, PFC extracts were examined to measure the coupling of erbB4 with NMDAR1, the obligatory subunit of NMDAR. As with PSD-95, the association of erbB4 with NMDAR1 was significantly enhanced in schizophrenia (t(13)=10.19, P<0.001; FIG. 9), indicating an increases in the modulation of NMDAR function by NRG1 stimulation in this disorder. Prefrontal cortex tissues were stimulated with 10 μM NMDA+1 μM glycine, and NMDAR activation was assessed by tyrosine phosphorylation of NMDAR2A (FIG. 9E) and the recruitment of PIPLCγ or nNOS (FIG. 9 F, G) by NMDAR1. In contrast, NMDA stimulation did not enhance tyrosine phosphorylation of erbB4 (FIG. 9 H).

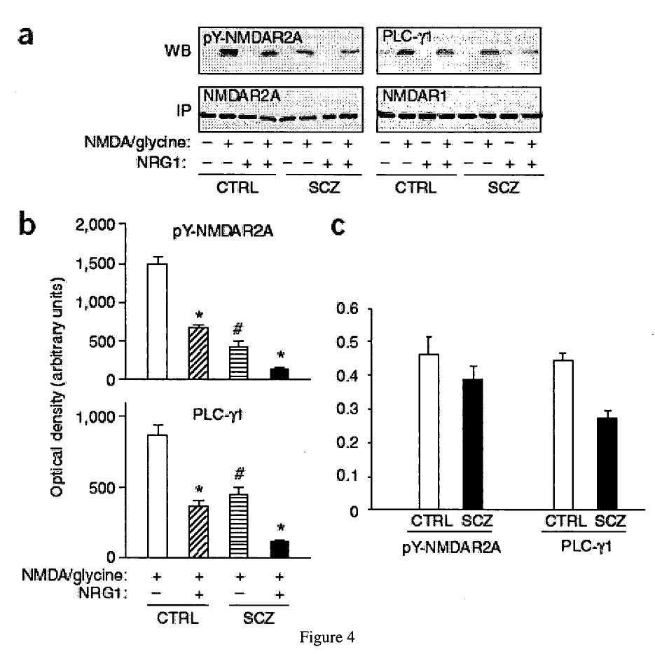

Evidence is accumulating that in rodent brain cells, NRG1 stimulation decreases NMDA-mediated ionic currents within minutes. If this is also the case in human PFC, then the enhanced NRG1-erbB4 signaling observed in schizophrenia could result in a further decrease in NMDAR function. To test this, the effects of NMDA stimulation in slices of PFC were examined from ten matched subject pairs using (i) vehicle only, (ii) NMDA (100 mM)+glycine (1 mM), (iii) NRG1 (200 ng/ml) or (iv) NMDA+glycine+NRG1. The changes from baseline in the tyrosine phosphorylation of NMDAR2A and the recruitment of phosphotidyl inositol phospholipase C-γ1 (PIPLC-γ1) were measured using the coimmunoprecipitation protocol (FIG. 4).

### Example 6

### PSD Fractions can be Reliably Isolated from Postmortem Brains of Human Subjects

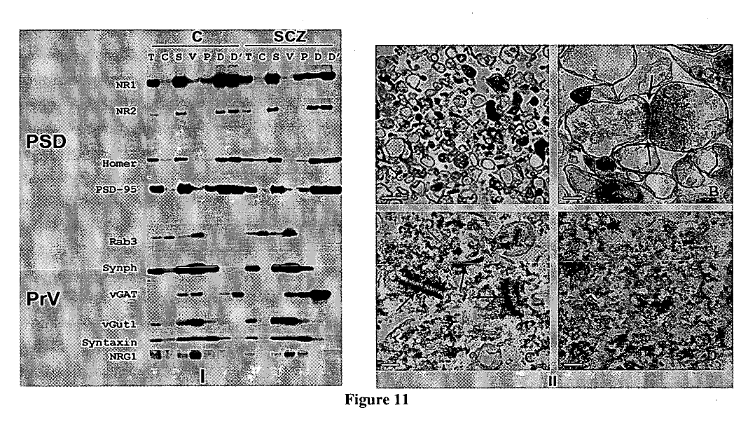

As shown in FIG. 11; postmortem PFC tissues from control and SCZ subjects were fractionated by several methods, using pH based differential extraction of synaptic membranes. (I) Various fractions from subcellular fractionation of the PSD from postmortem brain tissues whereby the PSD fraction were analyzed by immunoblotting for proteins highly enriched in either the PSD or presynaptic vesicles. (II) Electron micrographs of osmicated insoluble pellets obtained by immunoprecipitating the pellets overnight with anti-NMDAR1 and mixing with of agarose-conjugated protein A/G beads. Solubilized immunoprecipitates were size fractionated, and immuno-blotted with anti-PSD-95, homer, and other binding partners of NMDAR subunits were carried out. A, B: Synaptosomal extracts. They show surprisingly intact synaptic membranes and synaptic vesicles (II-A,B) as well as filamentous cross-bridges (see arrows in II-B). C: Note that presynaptic specialization is removed and that the PSD is thinner than shown in A and B. Abbrevations: T: total tissue extracts, C: cellular extracts, S: synaptic membranes, V: presynaptic vesicle, P: presynaptic membrane, D: PSD isolated by the method 1, D′: PSD isolated by the method 2. PrV: presynaptic vesicular fractions, NR: NMDA receptor, Synph: synaptophorin, vGAT: vesicular GABA transporter, vGlut1: vesicular glutamate transporter-1.

### Example 7

### erbB4 or NMDAR Signaling Proteins are not Altered in S/T, SPM, or PSD Fractions derived from the PFC of SCZ Subjects

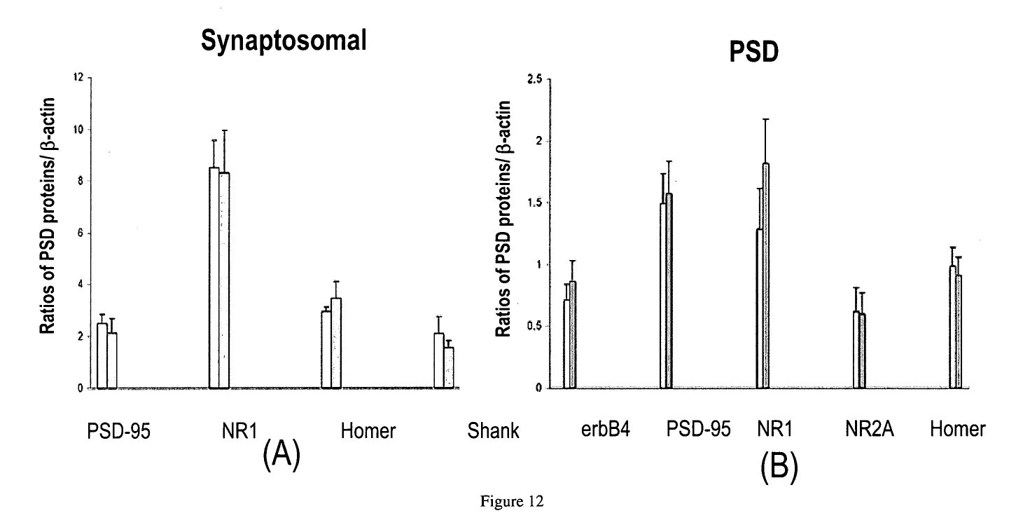

As shown in FIG. 12, PSD proteins in synaptosomal or PSD fractions were not dysregulated in the PFC of SCZ subjects compared with matched controls. (A, B) Postmortem PFC tissues from control and SCZ subjects were fractionated and analyzed for proteins by immunoblotting. (A) Proteins in synaptosomal fractions were normalized with respect to those of b-actin. (B) Proteins in PSD fractions were normalized with respect to those of b-actin. No significant differences were found between SCZ and control groups for the molecules examined in synaptosomal as well as in PSD fractions.

### Example 8

### In the PSD, the Association of PSD-95 with erbB4 or with NMDAR Subunits is Enhanced in the PFC of SCZ Subjects

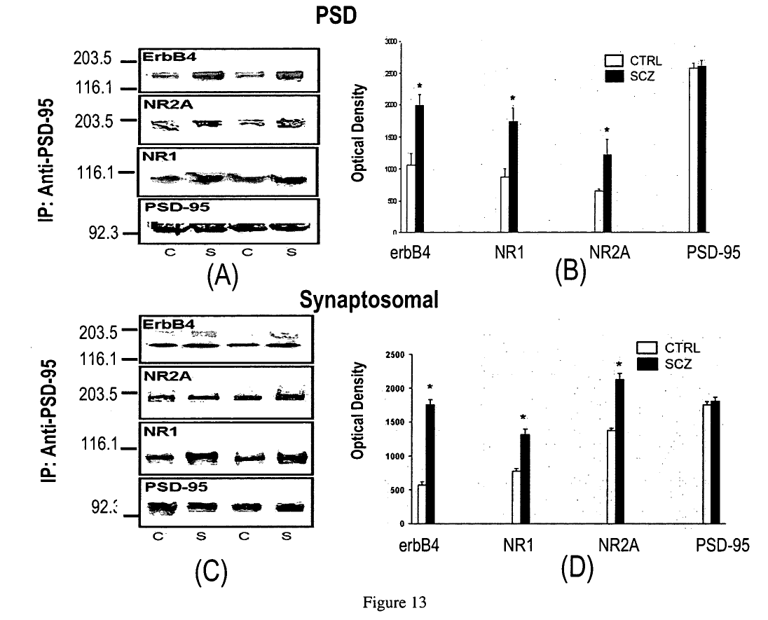

As shown in FIG. 13; Protein-protein associations were significantly altered in the PSD fractions derived from the PFC of SCZ subjects. (A, B) show PSD fractions of 12 pairs of CTRL and SCZ subjects that were immunoprecipitated for PSD-95. (A) A representative immunoblot. (B) Quantification of IP results. (C, D) Synaptosomal fractions of 12 pairs of control and SCZ subjects were immunoprecipitated for PSD-95. (C) A representative immunoblot. (D) Quantification of IP results.

### Example 9

### Associations among NR-1, PSD-95 and NR2A show a Trend Toward Dysregulations in the PSD Fractions of SCZ Subjects

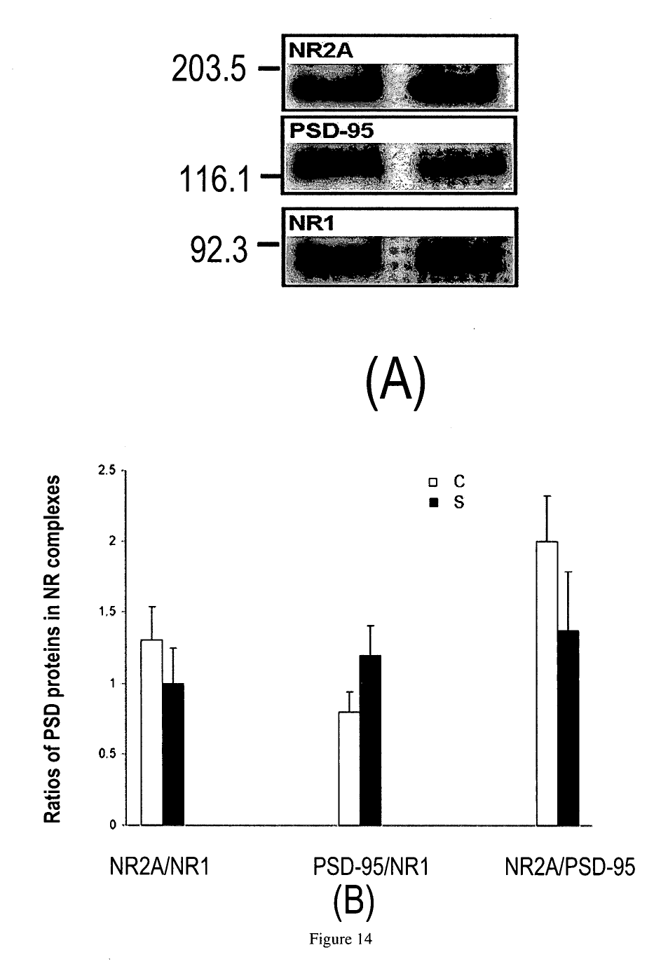

As shown in FIG. 14; Protein-protein associations trends towards dysregulation in PSD fractions of the PFC of SCZ subjects. (A, B) PSD fractions of 10 pairs of CTRL and SCZ subjects were immunoprecipitated for NR1. (A) A representative immunoblot. (B) Quantification of IP results.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of treating a neuropsychiatric disorder in a subject, comprising administering to said subject an agent capable of inhibiting the function of a Neurgulin-1 gene or its encoded or regulated proteins in said subject, whereby said Neurgulin-1 gene stimulates erbB4 signaling.

2. The method of claim 1, whereby inhibiting the function of a Neurgulin-1 (NRG1) gene or its encoded proteins, comprises lowering the level of a protein or a nucleic acid regulating the function of said Neurgulin-1 (NRG1) gene, or its encoded or regulated proteins.

3. The method of claim 1, whereby the regulated function is the modulating of erbB pathway.

4. The method of claim 1, whereby said agent is a siRNA, polyamides, triple-helix-forming agents, antisense RNA, synthetic peptide nucleic acids (PNAs), agRNA, LNA/DNA copolymers, small molecule chemical compounds, or a combination thereof.

5. The method of claim 1, whereby the agent is an antibody or fragment thereof, specific against the protein encoded by the Neuregulin-1 (NRG1) gene or a protein regulated by the expression of NRG1.

6. The method of claim 5, whereby the antibody or fragment thereof is specific for erbB4, PSD95, AKT, ERK2, NMDAR or a combination thereof.

7. The method of claim 5, whereby the antibody fragment is Fab, Fab′, Fab1, Fab2, Fc or scFv.

8. The method of claim 1, further comprising stimulating NMDAR function.

9. The method of claim 1, further comprising inhibiting the binding of erbB4 and PSD95.

10. The method of claim 1, whereby treating comprises reducing incidence, reducing symptoms, increasing relapse time or a combination thereof.

11. The method of claim 1, whereby treating comprises curing.

12. The method of claim 1, further comprises administering an additional antipsychotic agent.

13. A composition for the treatment of a neuropsychiatric disorder in a subject, comprising an agent capable of inhibiting the function of a Neurgulin-1 gene in said subject, whereby said Neurgulin-1 gene stimulates ErbB4 signaling, which further attenuates NMDAR hypofunction.

14. The composition of claim 13, wherein inhibiting the function of a Neurgulin-1 (NRG1) gene or its encoded proteins, comprises lowering the level of a protein or a nucleic acid regulating the function of said Neurgulin-1 (NRG1) gene, or its encoded or regulated proteins.

15. The composition of claim 13, wherein said agent is a siRNA, polyamides, triple-helix-forming agents, antisense RNA, synthetic peptide nucleic acids (PNAs), agRNA, LNA/DNA copolymers, small molecule chemical compounds, or a combination thereof.

16. The composition of claim 13, wherein the agent is an antibody or fragment thereof, specific against the protein encoded by the Neuregulin-1 (NRG1) gene or a protein regulated by the expression of NRG1.

17. The composition of claim 16, wherein the antibody or fragment thereof is specific for ErbB4, PSD95, AKT, ERK2, NMDAR or a combination thereof.

18. The composition of claim 16, wherein the antibody fragment is Fab, Fab′, Fab1, Fab2, Fc or scFv.

19. The composition of claim 13, further comprising an agent capable of stimulating NMDAR function.

20. The composition of claim 13, further comprising an agent capable of inhibiting the binding of erbB4 and PSD95.

21. A method of assessing erbB4 signaling in a prefrontal cortex of a subject having a neuropsychiatric disorder, comprising the step of stimulating postmortem brain tissues of said subject with an effective amount of Neuregulin-1 (NRG1), thereby enhancing tyrosine phosphorylation of erbB4.

22. The method of claim 21, whereby erbB4 signaling stimulation results in modulation of erbB4-PSD95 binding.

23. The method of claim 21, further comprising attenuating NMDAR function.

24. A method of screening for an agent capable of postmortem stimulation of a brain tissue, comprising the steps of: slicing frozen brain tissue; gradually thawing the frozen tissue; preparing a cell extract of the sliced tissue; contacting the cell extract with the agent; and immnuopercipitating the agent, wherein immunoblotting will indicate the ability of the agent to stimulate the tissue.

25. The method of claim 1, or 21, whereby the neuropsychiatric disorder is schizophrenia, bipolar disorder, or their combination.

26. The composition of claim 13, wherein the neuropsychiatric disorder is schizophrenia, bipolar disorder, or their combination.
